(19) United States
(12) Patent Application Publication
(10) Pub. No.: US 2010/0291603 A1
(43) Pub. Date: Nov. 18, 2010

(54) ASSESSING BLOOD COMPONENTS

(76) Inventors: Whyte G. Owen, Rochester, MN (US); Dong Chen, Rochester, MN (US); Christine Daigh, Middleton, WI (US)

Correspondence Address: FISH & RICHARDSON P.C., PO BOX 1022, MINNEAPOLIS, MN, 55440-1022, US

(21) Appl. No.: 12/532,453

(22) PCT Filed: Mar. 28, 2008
(86) PCT No.: PCT/US08/58708

§ 371 Date: Jun. 21, 2010

Related U.S. Application Data
(60) Provisional application No. 60/921,189, filed on Mar. 30, 2007.

(51) Int. Cl. C12Q 1/56; C40B 40/02

(52) U.S. Cl. 435/13; 506/14

(57) ABSTRACT

This document relates to methods and materials involved in assessing blood components (e.g., assessing von Willebrand factor activity or platelet activity) in mammals. For example, methods and materials involved in using labeled platelets to assess von Willebrand factor activity in a sample (e.g., plasma) from a mammal (e.g., a human) are provided.

### CROSS-REFERENCE TO RELATED APPLICATIONS

This application claims the benefit of U.S. Provisional Application Ser. No. 60/921,189, filed Mar. 30, 2007, hereby incorporated by reference in its entirety.

### STATEMENT AS TO FEDERALLY SPONSORED RESEARCH

This invention was made with government support under grant numbers HL078638 and HL083141 awarded by National Institutes of Health. The government has certain rights in the invention.

### BACKGROUND

1. Technical Field

This document relates to methods and materials involved in assessing blood components (e.g., assessing von Willebrand factor activity and platelet activity) in mammals. For example, this document relates to methods and materials involved in using labeled platelets to assess von Willebrand factor activity in a sample (e.g., plasma) from a mammal (e.g., a human).

2. Background Information

Von Willebrand disease (VWD) is caused by either a quantitative or qualitative deficiency of von Willebrand Factor (VWF; Sadler et al., J Thromb Haemost., 4 (10):2103-14 (2006); Schneppenheim and Budde, Hamostaseologie, 24:27-36 (2004); Sadler et al., Thromb Haemost.; 84:160-174 (2000)). The revised classification of VWD identifies three types of abnormalities. Type 1 VWD is characterized by a partial deficiency of VWF in plasma with proportional decreases in VWF activity. Type 3 VWD denotes an absence or trace amount of VWF in plasma. Type 2 VWD defines the qualitative abnormalities of VWF that have been further classified into four major subtypes. Type 2A VWD is characterized by the absence of high molecular weight VWF multimers in plasma and decreased VWF binding to platelets (Layergne et al., Br J Haematol., 82:66-72 (1992)). Type 2B VWD is also characterized by a loss of the largest plasma VWF multimers secondary to an increased affinity for platelet GPIb-IX-V complex (Takahashi and Shibata, Thromb Haemost., 52:267-270 (1984)). Type 2M VWD exhibits decreased VWF-dependent platelet functions in the presence of apparently normal VWF multimers (Mancuso et al., Blood, 88:2559-2568 (1996)). Type 2N (Normandy) VWD is characterized by a functional defect in binding to coagulation factor VIII (Mazurier et al., Br J Haematol., 88:849-854 (1994)).

### SUMMARY

This document provides methods and materials involved in assessing blood components in mammals (e.g., humans). For example, this document provides methods and materials involved in using labeled platelets to assess von Willebrand factor activity in a sample (e.g., plasma) from a mammal. In some cases, the methods and materials provided herein can include using platelets labeled with a first label and platelets labeled with a second label to assess von Willebrand factor activity in a sample (e.g., plasma) from a mammal. Having the ability to assess von Willebrand factor activity with sensitivity, specificity, and throughput can allow clinicians to identify patients having von Willebrand disease accurately and efficiently. As described herein, having the ability to assess von Willebrand factor activity also can allow clinicians to distinguish between types of von Willebrand disease and determine whether or not treatments for von Willebrand disease are effective. This document also provides methods and materials involved in using labeled platelets to assess platelet activity in mammals, which can allow clinicians to identify patients having conditions related to platelet function, such as coronary artery disease.

In general, one aspect of this document features a method for assessing von Willebrand factor activity. The method comprises, or consists essentially of: (a) contacting a plasma sample from a mammal with platelets labeled with a first label and platelets labeled with a second label, and (b) determining whether or not a complex is formed comprising a platelet labeled with the first label and a platelet labeled with the second label, wherein formation of the complex corresponds to the von Willebrand factor activity. The mammal can be a human or a dog. The sample can be plasma obtained from a fasting mammal. The first and the second label can be fluorescent labels. The determining step can comprise using flow cytometry. The contacting step can be performed in the presence of ristocetin.

In another aspect, this document features a method for assessing platelet activity in a mammal. The method comprises, or consists essentially of: (a) contacting collagen with platelets from the mammal labeled with a first label and platelets from the mammal labeled with a second label, and (b) determining whether or not a complex is formed comprising a platelet labeled with the first label and a platelet labeled with the second label, wherein the formation of the complex corresponds to the platelet activity. The mammal can be a human or a dog. The platelets can be obtained from a fasting mammal. The first label and the second label can be fluorescent labels. The determining step can comprise using flow cytometry. The collagen can be fibrous collagen.

In another aspect, this document features a composition comprising, or consisting essentially of, platelets labeled with a first label and platelets labeled with a second label. The first label and the second label can be fluorescent. The first label can fluoresce green, and the second label can fluoresce red. The platelets can be human platelets. The platelets can be platelets obtained from a fasting mammal.

In another aspect, this document features an assay plate comprising wells, wherein a surface of the well comprises an adhesive and a layer of platelets labeled with a label. The plate can be a 96-well plate. The label can be a fluorescent label.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is an illustration of green (lighter gray) and red (darker gray) fluorochrome labeled platelets binding to VWF multimers. An illustration agarose gel electrophoresis of VWF multimers is on the right. FIG. 1B is a plot illustrating the predicted results of unbound platelets and platelet micro-aggregates by flow cytometry. The unbound green and red fluorescence labeled platelets are at the upper left and lower right quadrants, respectively. The platelet micro-aggregates are at the upper right quadrant. FIG. 1C is an illustration of the experimental design. Plasma (2 μL) is added to the reaction tube containing fluorescent platelets and 1 mg/mL ristocetin prior to analysis by flow cytometry.

FIG. 2 contains plots of flow cytometry data obtained by analyzing VWF and ristocetin dependent platelet micro-aggregates. Buffer or 50% normal pooled plasma (NPP) was added to the reaction mixture and incubated at room temperature for 45 minutes. FIG. 2A is a plot of forward (FSC) and side light scattering (SSC) of the platelets. FIG. 2B is an FL1 versus FL3 plot of the platelets without NPP. FIG. 2C is an FL1 versus FL3 plot of the platelets and micro-aggregates with 50% NPP. Green: green only events (bottom right side events), Red: red only events (left side events), and Blue: double positive events (top right side events). FIG. 2D is an FSC versus FL1 plot of platelets and micro-aggregates with 50% NPP. Green: green only events (bottom right side events), Red: red only events (left side events), and Blue: double positive events (top right side events).

FIG. 3A is a histogram of green and red platelets and micro-aggregates. 50% NPP was included in the reaction. Green only (green), red only (red) and double positive micro-aggregates (blue; shifted to the left) were gated and plotted as a histogram in comparison with the green only events from the reaction without NPP (black). FIGS. 3B and 3C contain fluorescence microscopy images of the reaction mixture of 0% NPP (panel B) and 50% NPP (panel C). The inset for FIG. 3C represents a distinct green signal sandwiched between two distinct red signals. These images were captured at 400× magnification.

FIG. 4 contains data plots from flow cytometry tests performed using a series of NPP dilutions from 150 to 0%. FIG. 4A is a histogram is of the red events gated for green. The order of each designation in the key from top to bottom matches the line of the left peak from top to bottom. FIG. 4B is graph plotting double positive/total green events multiplied by 100 versus the relative plasma concentrations. Standard curves were obtained in the presence of 0, 0.5, 1.0, and 1.5 mg/mL ristocetin. FIG. 4C is a standard curve with 1.0 mg/mL of ristocetin plotted with the log scale of relative NPP concentration (150-3.125%). The R² equals 0.997.

FIG. 5 is a graph plotting the VWF:RCo level in a standard reaction performed with 100% NPP (NPP), a reaction in which ristocetin was absent from the reaction buffer (No Ristocetin in Reaction), a reaction in which ristocetin was absent from the dilution buffer (No Ristocetin in Diluent), a reaction in which the pH of the reaction buffer was 6.4 (pH 6.4), a reaction containing 0.5 μg/mL of non-immune mouse IgG (IgG), and a reaction containing 0.5 μg/mL of monoclonal antibody GTI-V3P (TGI-V3P).

FIG. 6A is a graph plotting platelet aggregation versus VWF:RCo (%). FIG. 6B is a graph plotting VWF:RCo levels in plasma samples from 51 healthy donors and 16 type 1 VWD patients analyzed by flow cytometry method, and VWF:RCo levels in plasma samples from 19 healthy donors and the same 16 samples from type 1 VWD patients determined by the platelet agglutination method. The VWF:RCo levels from both methods were plotted against the VWF:Ag levels.

FIG. 7 is a graph plotting VWF:RCo activity in samples from 51 normal donors, 16 type 1 VWD patients, and 20 type 2 VWD patients, measured using the flow cytometry or agglutination method (19 normal donors). The VWF:RCo/VWF:Ag ratios were calculated and plotted by the diagnostic classification including normal donor, type 1 and 2 VWD. The type 2 VWD samples were further grouped into 2A, 2B and 2M. The short horizontal bar indicates the mean value for each group. Two samples from type 2A and 2M VWD patients had less than 12.5% VWF:RCo activity by the agglutination method; therefore, the VWF:RCo/VWF:Ag ratios were not calculated.

### DETAILED DESCRIPTION

This document provides methods and materials related to assessing blood components in mammals. For example, this document provides methods and materials involved in using labeled platelets to assess von Willebrand factor activity in a sample (e.g., plasma) from a mammal. In some cases, the methods and materials provided herein can include using platelets labeled with a first label and platelets labeled with a second label to assess von Willebrand factor activity in a sample (e.g., plasma) from a mammal. As disclosed herein, a sample (e.g., plasma) from a mammal can be incubated with platelets labeled with a first label and platelets labeled with a second label, and molecules having the ability to promote platelet aggregation (e.g., VWF, ristocetin, collagen, fibrinogen, platelet surface glycoproteins, or antibodies). It can be determined whether or not complexes are formed comprising platelets labeled with the first label and platelets labeled with the second label, and the formation of such complexes can correspond to VWF activity and concentration.

Blood components (e.g., VWF activity) can be assessed in any mammal including, without limitation, a human, dog, cow, rodent, pig, horse, cat, or goat. Any appropriate sample (e.g., plasma) can be used to assess blood components, and any appropriate method can be used to obtain a sample from a mammal. For example, a blood sample (e.g., a fasting blood sample) can be obtained from a mammal by peripheral venipuncture. Plasma can be prepared from a blood sample using any standard method. For example, a blood sample can be drawn into a tube containing an anticoagulant (e.g., heparin or sodium citrate). The blood sample can be centrifuged at about 1,500×g for about 10 minutes at about 5° C. The upper phase (platelet poor plasma) can be aliquotted into tubes and can be used immediately or can be stored (e.g., at −70° C.) prior to being used.

Once a sample (e.g., plasma) is obtained from a mammal, the sample can be analyzed for the activity of a blood component (e.g., VWF). For example, a portion of a sample can be added to a solution containing platelets labeled with a first label, platelets labeled with a second label, and molecules that promote platelet aggregation in the presence of VWF (e.g., ristocetin, collagen, fibrinogen, platelet surface glycoproteins, or antibodies). The solution can contain additional components, such as buffer (e.g., imidazole saline buffer) and polypeptide (e.g., bovine serum albumin) to prevent adhesion of other polypeptides to the reaction tube. The reaction can be incubated (e.g., for 45 minutes at room temperature with rotation), diluted, and analyzed for formation of complexes comprising platelets labeled with the first label and platelets labeled with the second label.

Platelets can be prepared using any appropriate method. For example, a blood sample can be drawn into a tube containing an anticoagulant (e.g., heparin or sodium citrate). The blood sample can be centrifuged at about 150×g for about 15 minutes at room temperature. The upper phase (platelet rich plasma) can be transferred to another tube, and 1/10 volume of anticoagulant (e.g., ACD) can be added. The platelets can be counted, and the volume of the platelet rich plasma can be adjusted to achieve a suitable concentration of platelets (e.g., 500,000 platelets/μL.

Platelets (e.g., platelets present in platelet rich plasma) can be labeled with two different labels. Any appropriate labels can be used, such as cell permeating, fluorescent labels. In some cases, a label that fluoresces green and a label that fluoresces red can be used. In some cases, Mitotracker Red and CMFDA, or a comparable label, can be used.

Any appropriate method can be used to label platelets. For example, platelet rich plasma can be incubated with a cell permeating, fluorescent label. Stained platelets can be collected by centrifugation, washed, resuspended in buffer, and fixed (e.g., in formalin). Labeled platelets can be prepared on a large scale and stored at 4° C. for at least three months. For example, a batch of platelets can be prepared from a sample of blood obtained from one mammal, or from multiple samples of blood obtained from more than one mammal (e.g., more than one healthy mammal).

Any molecules that promote platelet activity (e.g., platelet aggregation) can be used and/or assessed. For example, VWF, ristocetin molecules, collagen molecules, fibrinogen, platelet surface glycoproteins, antibodies, or a combination thereof, can be used. The collagen molecules can be fibrous soluble collagen molecules. In some cases, shear stress, collagen, or other agonists (e.g., ADP) can be used to stimulate platelet activity.

The activity of a blood component (e.g., VWF) can be assessed by determining whether or not complexes are formed in a solution containing platelets labeled with a first label, platelets labeled with a second label, and molecules that promote platelet aggregation in the presence of VWF (e.g., ristocetin and/or collagen molecules). The presence of complexes comprising platelets labeled with a first label and platelets labeled with a second label can be detected using any appropriate method. For example, flow cytometry can be used to detect complexes comprising platelets labeled with a first label and platelets labeled with a second label, as well as individual platelets labeled with either the first or the second label. Flow cytometry also can detect the number of complexes (e.g., events) having both labels, the number of platelets (e.g., events) having a single label, the size distribution of labeled complexes, and the size distribution of platelets having a single label.

The formation of complexes can correspond to VWF activity and concentration. For example, the number of complexes (e.g., events) comprising a first and a second label (e.g., a label that fluoresces red and a label that fluoresces green), divided by the number of platelets (e.g., events) comprising only one label (e.g., a label that fluoresces green), and multiplied by 100 can correlate with VWF activity as well as VWF concentration. For example, a higher percentage of complexes having both labels can correlate with a higher VWF activity and also with a higher VWF concentration.

The ratio of the number of complexes (e.g., events) comprising a first and a second label (e.g., a label that fluoresces red and a label that fluoresces green) to the number of platelets (e.g., events) comprising only one label (e.g., a label that fluoresces green), multiplied by 100 can be compared to a standard curve to obtain a numerical value for VWF activity. A standard curve can be prepared using serial dilutions of normal pooled plasma as a source of VWF. In some cases, a standard curve can be prepared using a VWF reference standard (Bio/Data, Horsham, Pa.), or purified VWF. To generate a standard curve, different concentrations of VWF can be analyzed (e.g., using flow cytometry) in the presence of platelets labeled with a first label, platelets labeled with a second label, and molecules that can promote platelet aggregation in the presence of VWF to determine the number of complexes formed that contain both labels relative to the number of platelets containing one label. For each VWF concentration, the number of double positive complexes (e.g., events) can be divided by the number of platelets (e.g., events) positive for one label, and the value can be multiplied by 100. The percentages can be plotted against the log of the VWF concentration (e.g., concentration of normal pooled plasma) to generate a standard curve.

An assessment of VWF activity or concentration in a mammal can be used to determine whether or not the mammal has von Willebrand disease (VWD). In some cases, the VWF activity in a sample (e.g., plasma) from a mammal can be normalized to the VWF antigen level in the sample (e.g., plasma) from the mammal, and the ratio can be used to determine whether or not the mammal has VWD. In some cases, the VWF activity in a sample (e.g., plasma) from a mammal can be normalized to the VWF antigen level in the sample (e.g., plasma) from the mammal, and the ratio can be used to determine whether the mammal has type 1 or type 2 (e.g., type 2A) VWD. For example, a ratio of VWF activity to VWF antigen level that is less than about 0.5 can indicate that a mammal has type 2 VWD, and a ratio of VWF activity to VWF antigen level that is greater than about 0.5 can indicate that a mammal has type 1 VWD. In some cases, the VWF activity in a sample (e.g., plasma) from a mammal can be normalized to the VWF antigen level in the sample (e.g., plasma) from the mammal, and the ratio can be used to determine whether or not the mammal has type 2A VWD. For example, a ratio of VWF activity to VWF antigen level that is less than about 0.16 can indicate that a mammal has type 2 VWD and likely to be type 2A VWD, and a ratio of VWF activity to VWF antigen level that is greater than about 0.16 can indicate that a mammal is unlikely to have type 2A VWD. In some cases, the absence of detectable VWF activity can indicate that a mammal has type 3 VWD. VWF activity can be assessed using methods and materials described herein. VWF antigen level can be determined using any appropriate method. For example, VWF antigen level can be determined using an automated latex immunoassay, as described herein.

Methods and materials described herein can be used in combination with any other methods or materials to identify mammals having VWD. For example, methods and materials provided herein can be used in combination with a medical history, a test measuring the bleeding time it takes for blood to clot, a test measuring the level of factor VIII in the blood, and/or methods or materials described elsewhere (see, for example, Sadler et al., J Thromb Haemost., 4 (10):2103-14 (2006) and Schneppenheim and Budde, Hamostaseologie, 24:27-36 (2004)).

Methods and material described herein also can be used to determine whether or not a treatment for VWD is effective. For example, VWF activity can be assessed using any of the methods and materials provided herein prior to, during, and/or after being administered a treatment for VWD to determine whether or not the treatment is effective. An increase in VWF activity during or after treatment can indicate that the treatment is effective. A decrease, or no change, in VWF activity during or after treatment can indicate that the treatment is not effective. In some cases, an increase in a level of VWF activity in a mammal after administration of a therapy for VWD, as compared to the level before administration of the therapy, can indicate that the therapy is effective, whereas a decrease, or no change, in a level of VWF activity in a mammal after administration of a therapy for VWD, as compared to the level before administration of the therapy, can indicate that the therapy is not effective. In some cases, VWF activity can be normalized to VWF antigen level and used to determine whether or not a treatment for VWD is effective. An increase in the ratio during or after treatment, as compared to the ratio prior to treatment, can indicate that the treatment is effective, whereas a decrease in the ratio during or after treatment, as compared to the ratio prior to treatment, can indicate that the treatment is not effective.

Methods and materials disclosed herein also can be used to assess the length of VWF multimers in mammals. For example, flow cytometry can be used to determine the size distribution of complexes comprising platelets labeled with a first label and platelets labeled with a second label that were formed in the presence of VWF from a mammal (e.g., VWF in plasma from a mammal) and in the presence of molecules that can promote platelet aggregation in the presence of VWF. The size distribution of the complexes is proportional to the length of VWF multimers. The size distribution can be compared to a reference size distribution to determine the relative size distribution, or can be compared to a size distribution determined for the same mammal at an earlier time point to determine if the size distribution has changed. A reference size distribution can be generated using a VWF reference standard, using normal pooled plasma, or using plasma from patients having larger VWF multimers than normal. The length of VWF multimers can be monitored in any mammal, such as a human having atrial fibrillation.

Methods and materials disclosed herein also can be used to assess platelet activity in a mammal. Platelets (e.g., platelet rich plasma) can be obtained from a mammal and labeled using two different labels, as described above. The labeled platelets can be incubated with collagen molecules (e.g., fibrous soluble collagen molecules). The collagen molecules can be type III collagen molecules. In some cases, the collagen molecules can be type I and type III collagen molecules. After incubating the labeled platelets with collagen molecules, the platelets can be analyzed (e.g., using flow cytometry) for formation of platelet complexes comprising both labels. The presence of platelet complexes can indicate that the platelets are active, whereas the absence of platelet complexes can indicate that the platelets are not active. In some cases, a reduced level of formation of platelet complexes, or a reduced size distribution of platelet complexes, as compared the mean level or size distribution, respectively, of platelet complexes formed using platelets from healthy mammals can indicate that the platelets are dysfunctional. Such an assay can be used as a screening test for platelet dysfunction.

The methods and materials provided herein can be used for platelet functional testing, acquired VWD testing, AdamTS13 activity testing, and large VWF multimer testing. In some cases, the methods and materials provided herein can be used to monitor drug therapy for VWD.

In some cases, the methods and materials provided herein can be performed using a plate assay. For example, a plate assay can be performed to assess blood components. In some cases, a plate (e.g., a 96-well plate) can be coated with an adhesive (e.g., Elmer's Wonder Bond™, Archer Instant Bonding Adhesive™, Bondo Super Glue™, Duro Super Glue™, Scotch Instant Glue™, Instant Krazy Glue™, or CellTAK™ cell and tissue adhesive (BD Biosciences, Cat. No. 354240; Waite and Tanzer, Science, 212:1038-1040 (1981) and Waite, J. Biol. Chem., 258:2911-2915 (1983)) such that labeled platelets such as platelets labeled with a first label (e.g., green fluorescently labeled platelets) are attached to the plate. The adhesive can be deactivated such that subsequently added platelets do not adhere directly to the plate via the adhesive. For example, a pH sensitive adhesive such as CellTAK (BD Biosciences) can be used at a pH of 4-5 to adhere platelets. Subsequently, the pH can be adjusted to neutral such that the adhesive is no longer adhesive. Platelets labeled with a second label (e.g., red fluorescently labeled platelets) can be added to the plate together with a sample (e.g., plasma) obtained from a mammal to be tested. The conditions can be such that platelets labeled with a second label can form a complex with the platelets labeled with a first label with functional VWF is present in the sample. A fluorescence plate reader (e.g., Thermo device or Molecular device) can be used to determine whether or not particular wells of the plate have samples capable of forming duel color platelet complexes and, for example, indirectly measuring VWF ristocetin activity.

The invention will be further described in the following examples, which do not limit the scope of the invention described in the claims.

### EXAMPLES

### Example 1

### Determining von Willebrand Factor: Ristocetin Cofactor Activity Using Flow Cytometry

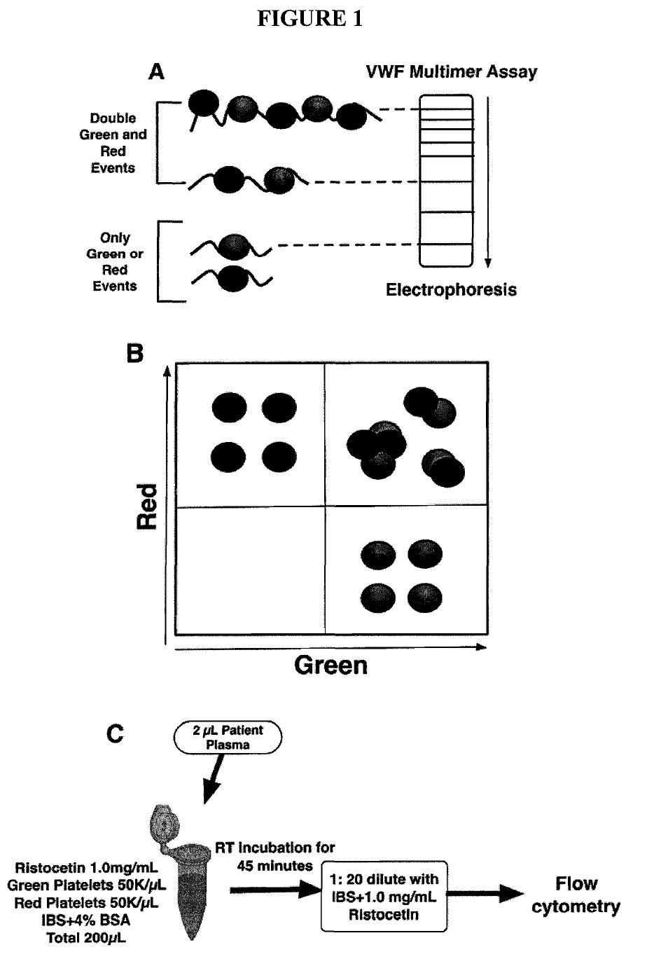

Assay design: Each VWF multimer has multiple binding sites for platelet membrane GPIb-IX-V complex. When both green and red fluorescent platelets bind to a single VWF molecule, a micro-aggregate forms (FIG. 1A). The micro-aggregates are detected by flow cytometry as the events positive for both green and red fluorescence (FIG. 1B). The extent of the micro-aggregate formation is affected by the binding affinity of VWF to platelets and the length of the VWF multimer. As illustrated in FIG. 1B, there are no double positive events if the VWF multimers are too short to bind at least two platelets. Based on this rationale, a new VWF:RCo method was designed (FIG. 1C). A source of VWF, such as normal pooled plasma (NPP) or a patient sample, is added to a tube containing green and red fluorescent platelets and ristocetin. The reaction is incubated at room temperature (20±2° C.) with oscillation at 15-20 rpm for 45 minutes while the binding of platelets to VWF equilibrates. The sample is then diluted and analyzed by flow cytometry.

Reagents and instruments: Fresh (less than 5 day-old) apheresis or random donor platelets were obtained from a blood bank. Imidazole, bovine serum albumin (BSA) fraction V, mouse IgG and all other chemicals were purchased from Sigma-Aldrich (St. Louis, Mo.). Ristocetin A sulphate was purchased from American Biochemical and Pharmaceutical Corporation (Marlton, N.J.). FACT-assayed NPP was purchased from George King Biomedical (Overland Park, Kans.). STA-LIATEST® vWF kit for VWF:Ag was purchased from Diagnostica Stago (Parsippany, N.J.). Mitotracker Red 580 (M22425) and CMFDA (C7025) were purchased from Invitrogen-Molecular Probes (Carlsbad, Calif.). Monoclonal antibody GTI-V3P was purchased from GTI diagnostics (Waukesha, Wis.). A BD FACS Calibur flow cytometer (BD Biosciences, San Jose, Calif.) was used for this study. An Olympus fluorescent microscope and Qcapture camera (Qimaging, Surrey, BC, Canada) were used for micrography.

Study population: After obtaining informed consent, a fasting blood sample (4.5 mL) was collected from each of 51 healthy volunteers and 36 VWD patients who were not taking any medication or receiving a blood product transfusion. Each blood sample was drawn into 1/9^{th} volume of 3.2% sodium citrate (0.5 mL). Platelet-poor plasma (PPP) was prepared by centrifugation at 1,500 g for 10 minutes at 5° C. Plasma samples were stored in small aliquots at −70° C. prior to testing. Criteria for diagnosis and typing of VWD were derived from the recommendations of the Scientific Standardization Committee on VWF of the International Society of Thrombosis and Haemostasis (Sadler et al., J Thromb Haemost., 4 (10):2103-14 (2006)). Clinical diagnosis of VWD was based on VWF:Ag, VWF:RCo, and a plasma VWF multimer assay.

Determination of VWF:Ag and VWF:RCo by agglutination: The automated latex immunoassay (LIA) was employed using the STA® Hemostasis System (Diagnostica Stago). Briefly, microlatex beads were coated with specific rabbit anti-human vWF antibody. In the presence of vWF antigen, the beads agglutinated and blocked light absorption. The change in optical density (OD) was proportional to the concentration of vWF antigen. Standard plasma pool dilutions and diluted test samples were tested in duplicate. VWF:Ag was automatically calculated by extrapolation from the calibration curve.

An assay for VWF:RCo based on platelet agglutination was derived from a method described elsewhere (Macfarlane et al., Thromb Diath Haemorrh., 34:306-308 (1975)). Fresh human donor platelets were obtained from a blood bank. The test was performed in a four-channel aggregometer (BioData, Horsham, Pa.). Fifty microliters of dilutions of normal or patient plasmas were added to 450 μL of washed platelets (250×10⁹/L) prepared in a silicone coated glass tube containing a stirring bar. After 15-20 seconds, the baseline curve was recorded, and then 50 μL of ristocetin solution (1 mg/mL final concentration) were added into the cuvette. The standard curve of a serial dilution of the NPP (50%, 25%, and 12.5%) was obtained by plotting, on a semilog scale, the slope of platelet agglutination measured from the maximum slope of each agglutination trace as a function of plasma dilution. The VWF:RCo of each sample was calculated by applying the test data to the standard curve. Definitive agglutination was usually seen with 12.5% NPP. Activity lower than 12.5% was reported as “<12.5%” instead of a value.

Preparation of fluorochrome labeled platelets: Platelet rich plasma (10 mL) with 500,000 platelets/μL was incubated with 10 μg of Mitotracker Red or 200 μg of CMFDA at 37° C. for two hours. Stained platelets were collected by centrifugation at 1125×g for 10 minutes at room temperature, and then washed four times with eight mL of HEPES-EDTA (20 mM HEPES, 142 mM NaCl, 15 mM KCl buffer, 2 mM Na₂EDTA, pH 7.4). Each platelet pellet was re-suspended in four mL of HEPES-EDTA and incubated at room temperature for 15 minutes. An equal volume (4 mL) of formalin fixative (1.37% formalin in HEPES-EDTA buffer) was added. The platelets were fixed at room temperature (in the dark) for 12 hours. Fixed platelets were further washed three times with imidazole saline (IBS) buffer (144 mM NaCl, 20 mM imidazole at pH 6.4). Platelets were counted and adjusted to 500,000/μL and stored at 4° C. for up to three months.

Determination of VWF:RCo by flow cytometry: Standard serial diluted normal pooled reference plasma or patient plasma (2 μL) were added to the reaction tubes containing IBS (pH 7.4), 4% BSA fraction V, 50,000/μL CMFDA and Mitotracker red fluorescence labeled platelets, and 1 mg/mL ristocetin. Reactions were incubated at room temperature for 45 minutes with 15-20 rpm vertical 360° rotation. The reaction mixture (20 μL) was diluted in 400 μL IBS containing 1 mg/mL ristocetin and analyzed immediately by flow cytometry. Platelets were gated first by forward and side light scattering (FSC/SSC). Green, red, and double stained populations were subsequently gated. A total of 50,000 green events (including the green only and double green/red events) were collected. The standard curve was plotted with the percentage of double positive vs. total green events multiplied by 100 against the log scale of normal pooled plasma concentration (%). The VWF:RCo of each sample was calculated by applying flow cytometry data to the standard curve. If the VWF:Ag was higher than 150%, 1 μL of the plasma was used. If the VWF:Ag was lower than 20%, 4 to 8 μL of plasma were used. In either case, VWF:RCo activities were calculated with the dilution factor.

Fluorescence microscopy: Flow cytometry reactions were performed as described above. A sampling of 10 μL reaction mixture was applied onto a glass slide and the fluorescent platelets were examined at 400× magnification. The Images of green and red fluorescent platelets were captured and overlayed using Photoshop 6.0 software.

### Results

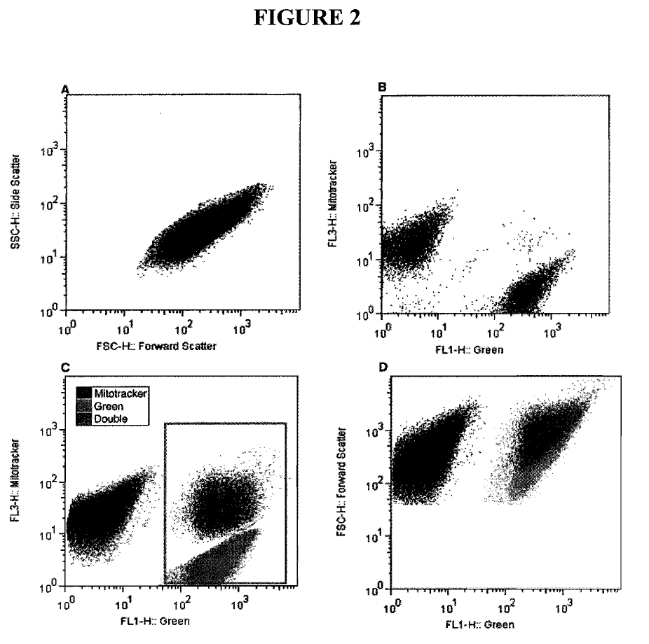

Fluorescent labeling of the reagent platelets: Platelets were labeled and adjusted to 500,000/μL as described. The fluorescent quality of labeled platelets was evaluated first. When equal concentrations of green and red platelets (50,000 platelets/μL) were mixed in the absence of plasma and ristocetin, the platelets showed a distribution typical of single platelets by forward (FSC) and side light scattering (SSC; FIG. 2A). The distinct green and red platelet populations were resolved by the FL1 and FL3 channel, respectively, and double positive events were insignificant (FIG. 2B).

Plasma and ristocetin dependent platelet micro-aggregate formation: A 1:100 dilution of NPP was arbitrarily set as 100% NPP (100% VWF:RCo activity). When 50% NPP was incubated with fluorescent platelets and one mg/mL ristocetin, the double positive (blue) events emerged in the right upper quadrant of the FL1/FL3 plot (FIG. 2C). To estimate the size of the micro-aggregates, the forward scatter was plotted against the FL1 data (FIG. 2D). The double positive particles appeared larger than the green only platelets.

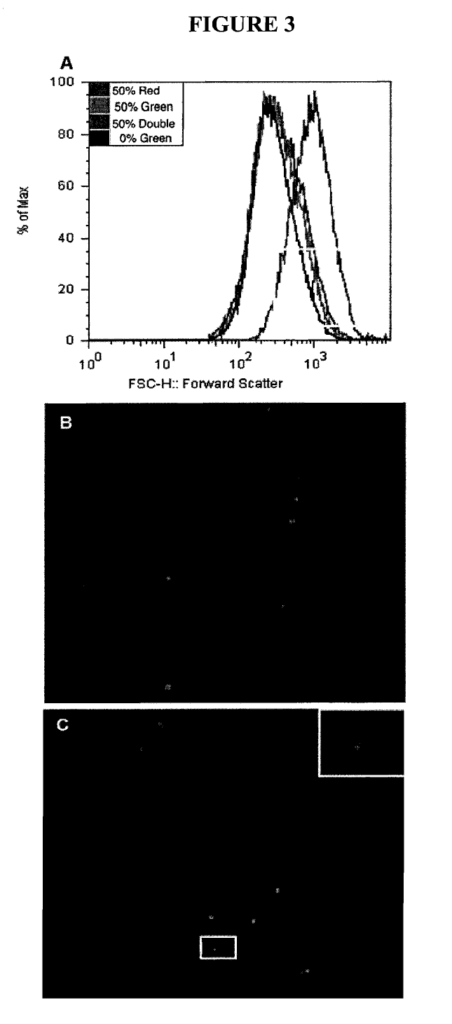

Flow cytometric characteristics of the micro-aggregates and the unbound platelets: FIG. 2D indicates that double positive particles are platelet micro-aggregates whereas green or red only events are mainly unbound single platelets. The majority of the platelets was presumed to be unbound when plasma was not present. The FSC histogram of double positive particles of the 50% NPP shifted to the right when it was compared to the green only events of 0% plasma (FIG. 3A). In contrast, the FSC histogram of the green and red only events of 50% NPP were similar to the green only events of 0% plasma (FIG. 3A).

To confirm this flow cytometry observation visually, fluorescent microscopy was performed on micro-aggregates that were formed with 50% NPP. When plasma was absent, green and red platelets were separated from each other, indicating minimal aggregation between green and red platelets (FIG. 3B). Micro-aggregates containing double fluorescent platelets, defined by close geometric proximity between green and red platelets, were observed when the plasma concentration was at 50% (FIG. 3C). These experiments confirmed that the double positive population represented the platelet micro-aggregates, while the single colored platelets were mainly unbound platelets.

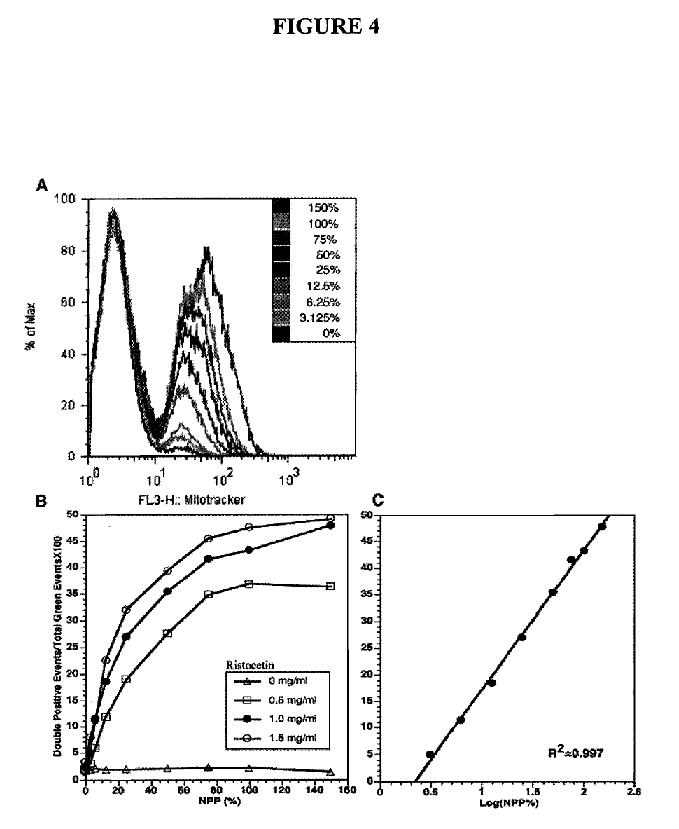

Quantification of VWF:RCo: With serial dilutions of NPP as a source of VWF, the total green events were first gated as shown in FIG. 2C (the green box) to encompass both double positive and green only populations. Histograms of the FL3 data of these green fluorescent platelets and micro-aggregates are shown in FIG. 4A. The unbound (green only population) platelets are represented by the first peak on the left, whereas the micro-aggregates are represented by the second peak on the right. The second peak declined with decreasing plasma concentration and reached the baseline when plasma was absent. It appeared that the percent of double positive events divided by total green events (D/G) correlated with VWF:RCo activity. Therefore, the quotient (D/G) was multiplied by 100 (D/G×100) and plotted against the plasma concentration (FIG. 4B). The curve reached a plateau at 150% NPP. When D/G×100 was plotted against the log scale of plasma concentration, a linear relationship was established with an R square value equal to 0.997 (FIG. 4C).

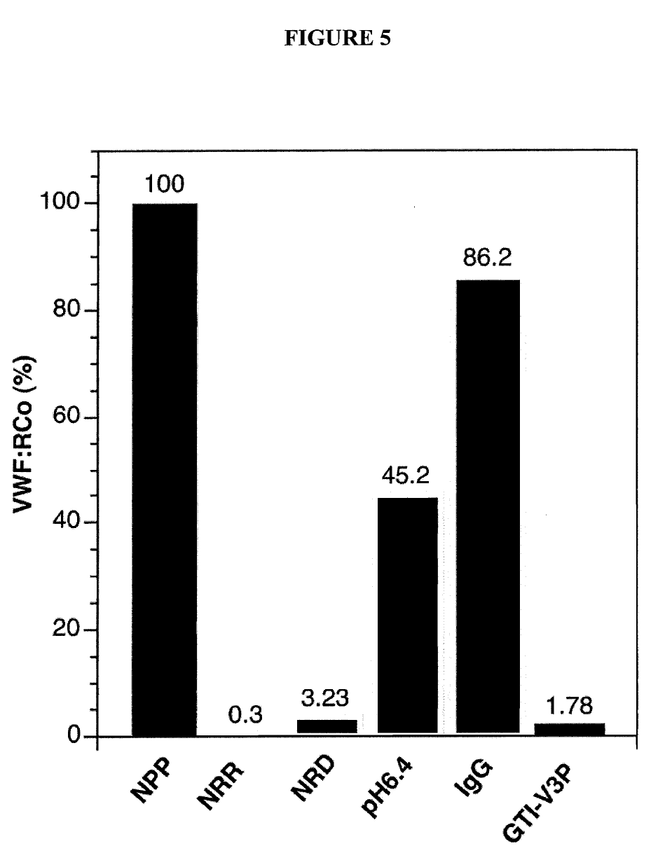

Specificity of the flow cytometry assay: The unique features of VWF dependent platelet micro-aggregate formation were used to confirm VWF specificity. The first feature is that VWF-platelet binding is ristocetin dependent. The standard curves with serial dilutions of plasma were performed using different concentrations of ristocetin (FIG. 4A). The micro-aggregate formation depended on the ristocetin concentration, and was maximized when the concentration of ristocetin reached one mg/mL. When ristocetin was not present (FIG. 5), VWF:RCo activity was significantly decreased from 100% to the baseline. The second feature is that VWF-platelet binding is sensitive to pH (Kao et al., J Clin Invest., 63:656-664 (1979)). Reducing the pH of the binding reaction to 6.4 resulted in a loss of 55% of the binding activity (FIG. 5). Finally, a monoclonal antibody, GTI-V3P, that specifically inhibits VWF A1 domain binding to GPIb-IX-V complex (Montgomery et al., Methods Enzymol., 121:702-717 (1986)) inhibited the micro-aggregate formation. These results indicated that the flow cytometry assay is VWF specific.

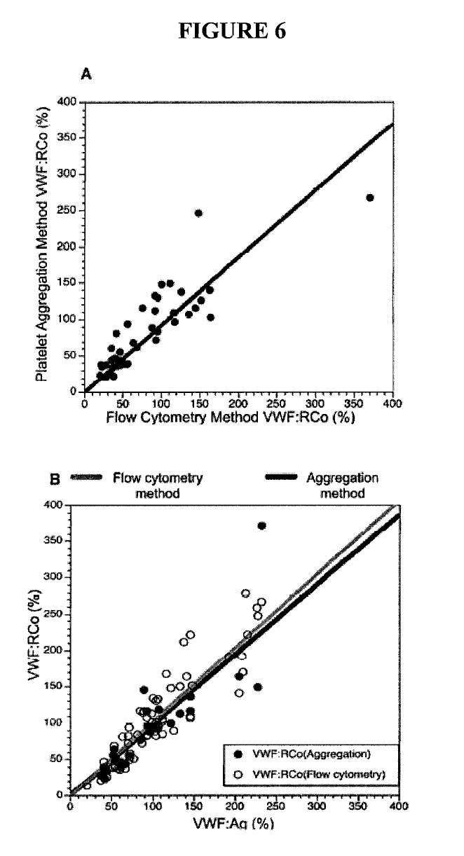

Quantification of VWF:RCo in samples from normal donors and type 1 VWD patients using flow cytometry: In samples from healthy donors and type 1 VWD patents, the VWF:RCo should be close to the VWF:Ag. Plasma samples from 51 healthy donors and 16 type 1 VWD patients were tested using the flow cytometry method, and plasma samples from 19 healthy donors and the same 16 samples from type 1 VWD patients were tested using the platelet agglutination method. Both methods had a linear correlation with VWF antigen level (FIG. 6). The correlation coefficient (r2) for the flow cytometry method was 0.82 (P<0.0001) compared to 0.72 (P<0.0001) for the platelet agglutination test. There was no significant difference between the two methods (Paired t-test, P=0.69).

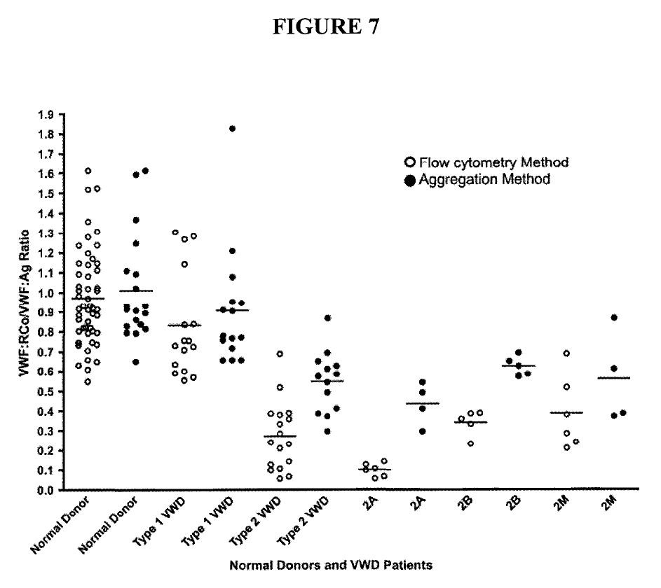

Detection of type 2 VWD using the flow cytometry method: Results obtained by analyzing samples from 51 normal donors, 16 type 1 VWD patients, and 20 type 2 VWD patients using the flow cytometry method were compared to results obtained by analyzing samples from 19 normal donors, 16 type 1 patients, and 20 type 2 patients using the platelet agglutination method (FIG. 7 and Table 1). The mean VWF:RCo/VWF:Ag ratio from normal healthy donors and type 1 VWD patients was close to 1.0 by both methods. In contrast, the mean VWF:RCo/VWF:Ag ratio of the samples from type 2 VWD patients was 0.25 by the flow cytometry method. This ratio was significantly lower than that of the samples from normal donors or type 1 VWD patients (P<0.0001). In comparison, the ratio by the platelet agglutination method was 0.52. Type 2 VWD patients were further divided into 2A, 2B, and 2M. Using the flow cytometry method, samples from 2A VWD patients gave the lowest VWF:RCo/VWF:Ag ratios (mean=0.11), which were significantly different from 2B (mean=0.29, P=0.0008) and 2M (mean=0.39, P=0.0016). In contrast, the difference among samples from the different type 2 VWD subgroups was less significant when determined by the agglutination method. These results indicate that the flow cytometry method detects type 2 VWD with high sensitivity.

TABLE 1
VWF:RCo/VWF:Ag ratios determined using the flow cytometry and agglutination methods.

| | Flow cytometry method | | | | | | Agglutination method | | | | | |
|---|---|---|---|---|---|---|---|---|---|---|---|---|
| | Normal | Type 1 | Type 2 | 2A | 2B | 2M | Normal | Type I | Type 2 | 2A | 2B | 2M |
| Number of values | 51 | 16 | 20 | 8 | 6 | 6 | 19 | 16 | 16 | 6* | 6 | 4* |
| VWF:Ag | | | | | | | | | | | | |
| Mean | 124.1 | 56.38 | 74.00 | 76.88 | 81.50 | 62.67 | 126.8 | 56.38 | 83.75 | 89.00 | 81.50 | 79.25 |
| Median | 107.0 | 57.50 | 53.00 | 62.50 | 53.00 | 37.00 | 108.0 | 57.50 | 63.50 | 74.00 | 53.00 | 78.50 |
| Std. Deviation | 48.04 | 11.33 | 50.57 | 43.43 | 68.88 | 45.78 | 48.12 | 11.33 | 52.16 | 43.90 | 68.88 | 48.90 |
| Maximum | 233.0 | 72.00 | 220.0 | 166.0 | 220.0 | 125.0 | 233.0 | 72.00 | 220.0 | 166.0 | 220.0 | 125.0 |
| Minimum | 58.00 | 37.00 | 28.00 | 36.00 | 40.00 | 28.00 | 58.00 | 37.00 | 35.00 | 51.00 | 40.00 | 35.00 |
| VWF:RCo | | | | | | | | | | | | |
| Mean | 125.5 | 50.06 | 16.44 | 9.250 | 16.31 | 26.17 | 127.9 | 49.56 | 44.50 | 34.33 | 49.67 | 52.00 |
| Median | 108.0 | 42.55 | 9.610 | 5.860 | 13.85 | 16.71 | 116.0 | 45.00 | 33.00 | 33.00 | 32.50 | 43.50 |
| Std. Deviation | 59.12 | 20.06 | 16.03 | 11.01 | 6.724 | 23.77 | 66.52 | 17.53 | 33.05 | 14.87 | 39.62 | 46.28 |
| Maximum | 278.3 | 93.80 | 64.60 | 36.30 | 26.80 | 64.60 | 371.0 | 88.00 | 128.0 | 60.00 | 128.0 | 108.0 |
| Minimum | 37.60 | 21.00 | 3.300 | 3.300 | 9.900 | 6.500 | 48.00 | 23.00 | 13.00 | 15.00 | 25.00 | 13.00 |
| VWF:RCo/Ag | | | | | | | | | | | | |
| Ratio | | | | | | | | | | | | |
| Mean | 0.97 | 0.88 | 0.25 | 0.11 | 0.29 | 0.39 | 1.00 | 0.95 | 0.52 | 0.40 | 0.62 | 0.56 |
| Median | 0.92 | 0.79 | 0.23 | 0.10 | 0.34 | 0.33 | 0.91 | 0.91 | 0.56 | 0.39 | 0.60 | 0.50 |
| Std. Deviation | 0.24 | 0.26 | 0.17 | 0.06 | 0.13 | 0.19 | 0.27 | 0.31 | 0.16 | 0.10 | 0.05 | 0.23 |
| Maximum | 1.60 | 1.30 | 0.69 | 0.22 | 0.39 | 0.69 | 1.60 | 1.80 | 0.86 | 0.54 | 0.69 | 0.86 |
| Minimum | 0.55 | 0.55 | 0.05 | 0.06 | 0.05 | 0.21 | 0.65 | 0.66 | 0.29 | 0.29 | 0.57 | 0.37 |

*Two samples from type 2A and 2M VWD patients had less than 12.5% VWF:RCo activity by the agglutination method; therefore, the VWF:RCo/VWF:Ag ratios were not calculated.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing von Willebrand factor activity, said method comprising:
(a) contacting a plasma sample from a mammal with platelets labeled with a first label and platelets labeled with a second label, and
(b) determining whether or not a complex is formed comprising a platelet labeled with said first label and a platelet labeled with said second label,
wherein formation of said complex corresponds to said von Willebrand factor activity.

2. The method of claim 1, wherein said mammal is a human or a dog.

3. The method of claim 1, wherein said sample is plasma obtained from a fasting mammal.

4. The method of claim 1, wherein said first and said second label are fluorescent labels.

5. The method of claim 1, wherein said determining step comprises using flow cytometry.

6. The method of claim 1, wherein said contacting step is performed in the presence of ristocetin.

7. A method for assessing platelet activity in a mammal, said method comprising (a) contacting collagen with platelets from said mammal labeled with a first label and platelets from said mammal labeled with a second label, and (b) determining whether or not a complex is formed comprising a platelet labeled with said first label and a platelet labeled with said second label, wherein the formation of said complex corresponds to said platelet activity.

8. The method of claim 6, wherein said mammal is a human or a dog.

9. The method of claim 6, wherein said platelets are obtained from a fasting mammal.

10. The method of claim 6, wherein said first label and said second label are fluorescent labels.

11. The method of claim 6, wherein said determining step comprises using flow cytometry.

12. The method of claim 6, wherein said collagen is fibrous collagen.

13. A composition comprising platelets labeled with a first label and platelets labeled with a second label.

14. The composition of claim 13, wherein said first label and said second label are fluorescent.

15. The composition of claim 14, wherein said first label fluoresces green and said second label fluoresces red.

16. The composition of claim 13, wherein said platelets are human platelets.

17. The composition of claim 13, wherein composition comprises ristocetin.

18. The composition of claim 13, wherein said platelets are platelets obtained from a fasting mammal.

19. An assay plate comprising wells, wherein a surface of said well comprises an adhesive and a layer of platelets labeled with a label.

20. The assay plate of claim 19, wherein said plate is a 96-well plate.

21. The assay plate of claim 19, wherein said label is a fluorescent label.
